(19) United States
(12) Patent Application Publication
(10) Pub. No.: US 2008/0051694 A1
(43) Pub. Date: Feb. 28, 2008

(54) Medical treatment equipment

(75) Inventor: Tomihisa Kato, Aichi-ken (JP)

Correspondence Address: MORGAN LEWIS & BOCKIUS LLP, 1111 PENNSYLVANIA AVENUE NW, WASHINGTON, DC, 20004, US

(73) Assignee: Asahi Intecc Co., Ltd.

(21) Appl. No.: 11/878,233

(22) Filed: Jul. 23, 2007

(30) Foreign Application Priority Data
Aug. 23, 2006 (JP) 2006-227187

(51) Int. Cl. A61B 17/20; A61B 17/44

(52) U.S. Cl. 604/22; 606/207

(57) ABSTRACT

In a medical treatment equipment (1), a sheath body (5) has a deformed tight coil structure in which a plurality of wire elements (7, 8) are formed triangular in cross section, and helically wound with one coil line turn alternately arranged in neighborhood of other coil line turn. An apex of the triangular cross section of the one coil line turn facing an inner diameter side of the sheath body 5, and an apex of the triangular cross section of the other coil line turn facing an outer diameter side of the sheath body 5. Lateral sides of neighboring triangular cross sections are relatively slidable each other at a mutual neighborhood side (9) in a radial direction when the sheath body (5) is bent. This prevents the sheath body (5) from developing a line gap (C) between coil line turns of the deformed tight coil structure, and protecting the sheath body (5) against a tensile load which would otherwise be applied when the sheath body (5) is curvedly bent within a somatic cavity. Further, the sheath body (5) enables an operator to delineate a clear supersonic image under a supersonic three-dimensional echo.

### BACKGROUND OF THE INVENTION

1. Field of the Invention

The invention relates to a medical treatment equipment which is inserted into a somatic cavity to collect cellular tissues upon implementing a treatment for a somatic body, and employed as a treatment device for an endoscope, a biopsy forceps and a multi-functional surgical treatment device.

2. Description of Related Art

In a medical treatment equipment for treating a somatic cavity, it is inserted through a thin and tortuous blood vessel by extracorporeally implementing a push-pull and rotational action manually at a proximal operation side through an operational portion.

During the implemention, the manual operation actuates a treatment portion at a distal end of the medical treatment equipment to surgically cure a diseased area of the somatic cavity. The medical treatment equipment generally has the treatment portion provided at a distal end side, the operational portion provided at a proximal end side, and an elongate core which connects the treatment portion to the operational portion. The elongate core is inserted to be placed within a flexible thin sheath body.

The sheath body of the medical treatment equipment is made by tightly winding a single wire circular in cross section with a predetermined diameter so as to form a tight knit coil structure.

By way of illustration, Japanese laid-open patent application No. 2001-017386 discloses a medical treatment device in which a long sheath body is made by tightly winding a single wire helically into a tight knit coil structure, to which an elongate core is inserted. Japanese laid-open patent application No. 2002-011017 discloses a forceps for use in an endoscope, Japanese laid-open patent application No. 10-290803 discloses a medical treatment device for use in an endocope, and Japanese laid-open patent application No. 2002-282261 discloses a multi-functional surgical treatment device. In each case of the three patent applications, there is provided the sheath body having a single wire helically wound into the tight knit coil structure.

Upon inserting the long sheath body into the sinuous blood vessel, the sheath body is subjected to a bending force at an outer side of the sheath body as a tensile load, and develops a line gap between coil line turns of the sheath body, so as to be curvedly deformed with the bending tensile stress remained in the sheath body.

The sheath body is subjected to the bending force at an inner side of the sheath body as a contractile load, so as to be curvedly deform with the coil line turns tightly knit while having the bending contractile stress remained in the sheath body.

For this reason, the sheath body subjects its bending portion to a restitutive force which urges the sheath body to return toward a straight-shaped configuration. The restitutive force makes the bending portion strongly engage against a vascular wall, so as to injure the vascular wall, while at the same time, obstructing the sheath body from advancing in the blood vessel.

The bending deformation lengthens a neutral line (central line) of the sheath body, and necessarily results in the sheath body being dimensionally lengthened. This accompanies a dimensional change on an effective action length of the elongate core which is placed within the sheath body, and exerting a strong tensile load to the elongate core, thus imparting a harmful action to the treatment portion while advancing the sheath body in the somatic cavity, and detering the operational portion from pushing, pulling and rotating the elongate core, so as to lose a good maneuverability necessary to attain an appropriate treatment.

Therefore, it is an object of the invention to overcome the above drawbacks, and provide a medical treatment equipment which is capable of preventing a sheath body from being lengthened in a somatic cavity, and avoiding a tensile load from being applied to an outer side of the sheath body when the sheath body is bent, and further delineating a clear supersonic image under a three-dimensional supersonic echo.

### SUMMARY OF THE INVENTION

According to the invention, there is provided a medical treatment equipment having a treatment portion provided at a distal end portion, an operational portion provided at a proximal portion, an elongate core provided to connect the treatment portion to the operational portion, and an elongated flexible sheath body, to which the elongate core is inserted.

The sheath body has a deformed tight coil structure in which a plurality of wire elements are formed triangular in cross section, and helically wound with one coil line turn alternately arranged in neighborhood of other coil line turn. An apex of the triangular cross section of the one coil line turn faces an inner diameter of the sheath body, and an apex of the triangular cross section of the other coil line turn, faces an outer diameter of the sheath body. One lateral side of the triangular cross section of the one coil line turn and one lateral side of the triangular cross section of the other coil line turn come in contact with each other to form a mutual neighborhood side. Lateral sides of neighboring triangular cross sections are relatively slidable each other at the mutual neighborhood side in a radial direction of the sheath body when the sheath body is bent.

With the tight knit coil structure provided in the sheath body of the medical treatment equipment, the plurality of wire elements triangular in cross section are alternately combined to form the deformed tight coil structure, as opposed to a tight knit coil structure in which a single wire ordinarily circular in cross section are helically wound simply. Characteristic of the invention is that the wire elements relatively slides at the lateral sides of neighboring triangular cross sections.

It is to be noted that the “deformed structure” herein means that the structure is different from an ordinary configuration, the “tight coil structure” means that the wire elements are closely wound with no line gap appeared between their coil line turns under the straight-shaped configuration with no load imposed on the sheath body.

From the reason that the wire elements relatively slides to be deformed at the lateral sides of neighboring triangular cross sections when the sheath body is bent in the somatic cavity, it is possible to prevent the sheath body from being lengthened, and avoiding a tensile load from being applied to an outer side of the sheath body with no line gap appeared between the coil line turns.

Although it is desirable to form a concave portion (reflective-echo area) on an outer surface of the sheath body in order to achieve a clear supersonic image under a three-dimensional supersonic echo, it is unnecessary to form the concave portion in the invention because the relative slide between the wire elements form an undulation (concave-convex area) on the outer surface of the sheath body with the concave portion served as the reflective-echo area.

According to other aspect of the invention, there is provided a medical treatment equipment having a treatment portion provided at a distal end portion, an operational portion provided at a proximal portion, an elongate core provided to connect the treatment portion to the operational portion, and an elongated flexible sheath body, to which the elongate core is inserted.

The sheath body has a deformed tight coil structure in which a plurality of wire elements are formed trapezoidal in cross section, and helically wound with one coil line turn alternately arranged in neighborhood of other coil line turn. An upper short side of the trapezoidal cross section of the one coil line turn faces an inner diameter side of the sheath body, and an upper short side of the trapezoidal cross section of the other coil line turn faces an outer diameter side of the sheath body. One oblique side of the trapezoidal cross section of the one coil line turn and one oblique side of the trapezoidal cross section of other coil line turn come in contact with each other to form a mutual neighborhood side. Oblique sides of neighboring trapezoidal cross sections are relatively slidable each other at the mutual neighborhood side in a radial direction of the sheath body when the sheath body is bent.

From the reason that the wire elements relatively slides to be deformed at the oblique sides of neighboring trapezoidal cross sections when the sheath body is bent in the somatic cavity, it is possible to prevent the sheath body from being lengthened in a somatic cavity, and avoiding a tensile load from being applied to an outer side of the sheath body with no line gap appeared between the coil line turns.

According to other aspect of the invention, there is provided a medical treatment equipment having a treatment portion provided at a distal end portion, an operational portion provided at a proximal portion, an elongate core provided to connect the treatment portion to the operational portion, and an elongated flexible sheath body, to which the elongate core is inserted.

The sheath body has a deformed tight coil structure in which a plurality of wire elements formed triangular in cross section and a plurality of wire elements formed trapezoidal in cross section are helically wound with one coil line turn alternately arranged in neighborhood of other coil line turn. An upper short side of the trapezoidal cross section of the one coil line turn faces an inner diameter side of the sheath body, and an apex of the triangular cross section of the other coil line turn faces an outer diameter side of the sheath body. One oblique side of the trapezoidal cross section of the one coil line turn and one side of the triangular cross section of the other coil line turn come in contact with each other to form a mutual neighborhood side. One oblique side of the trapezoidal cross section and the one side of the triangular cross section are relatively slidable each other at the mutual neighborhood side in a radial direction of the the sheath body when the sheath body is bent.

Such is the structure that the wire elements relatively slides at the oblique side of the trapezoidal cross section and the one side of the triangular cross section when the sheath body is bent in the somatic cavity, it is possible to prevent the sheath body from being lengthened, and avoiding a tensile load from being applied to an outer side of the sheath body with no line gap appeared between the coil line turns.

According to other aspect of the invention, cross sectional shapes of the plurality of wire elements are analogous each other. The analogous configuration is one of the examples as the cross sectional shape of the deformed tight coil structure.

According to other aspect of the invention, cross sectional shapes of the plurality of wire elements are identical each other. This makes it sufficient to produce a single type of common wire elements, thus improving the productivity with the manufacturing cost reduced.

According to other aspect of the invention, cross sectional shapes of the plurality of wire elements have equilateral oblique sides, and angles of the equilateral oblique sides are 45 degrees. This makes it possible to equipoise a dual component force along the equilateral oblique sides, thereby enabling the user to smoothly slide the neighboring wire elements in the radial direction when the sheath body is deformed from the straight-shaped configuration to the bend-shaped configuration and vice verse.

According to other aspect of the invention, the treatment portion is a biopsy cup which is operated by a push-pull manipulation through the operational portion for collecting cellular tissues.

With the sheath body prevented from being lengthened upon bending the sheath body in the somatic cavity, it is possible to actuate the biopsy cup to open and shut in the stable fashion upon collecting the cellular tissues even when the sheath body accumulates a greater amount of bends before reaching the biopsy cup to a target destination in the somatic cavity.

Namely, although the bending operation causes to curvedly bend the sheath body, the bending operation causes no shortage of an operational stroke (no operational shortage of the elongate core), thereby enabling the user to insure that the biopsy cup can quickly responds to the open and shut action of the operational portion.

According to other aspect of the invention, the treatment portion is a looped wire which is tightened to be diametrically reduced by pulling the elongate core through the operational portion for tying up the cellular tissues so as to form an endoscopic treatment structure.

With the sheath body prevented from being lengthened upon bending the sheath body, it is possible to conveniently obviate the looped wire from being tightened upon bending the sheath body in the somatic cavity. The bending operation accompanies no shortage of an operational stroke, thereby enabling the user to insure a quick response to the diametrically reducing action of the looped wire.

According to other aspect of the invention, the treatment portion is a clip retainer which accommodates a clip within the sheath body and retains the clip in cellular tissues by pulling the elongate core through the operational portion, so as to form a multi-functional surgical treatment structure.

With the sheath body prevented from being lengthened upon bending the sheath body, it is possible to protect the clip against positional variations within the sheath body upon bending the sheath body in the somatic cavity. The bending operation accompanies no shortage of an operational stroke, thereby enabling the user to insure a quick response to the retaining action of the clip.

According to other aspect of the invention, the treatment portion is an endoscope-angle shifting portion which is manipulatively altered by adjusting a tension of the elongate core through a rotational movement of the operational portion so as to form an endoscopic structure.

With the sheath body prevented from being lengthened upon bending the sheath body, it is possible to avoid the endoscope-angle shifting portion from unintentionally being varied upon bending the sheath body. The bending operation accompanies no shortage of an operational stroke, thereby enabling the user to insure a quick response to the changing action of the endoscope-angle shifting portion.

According to other aspect of the invention, a sensor is built in the sheath body to form a sensor-attached guide wire structure.

Devoid of the line gap between the coil line turns of the wire elements, it is possible to protect the sensor against a turbulance in the blood vessel which otherwise would be developed due to the line gap appeared between the coil line turns, so as to render output waves unstable when the sensor generates the output waves. With the sheath body prevented from being lengthened, it is possible to protect a lead wire against disconnection so as to insure a continued monitoring.

According to other aspect of the invention, the sheath body has a dual region structure which consists of a distal end side region having the deformed tight coil structure and a proximal end side region having a wire-stranded hollow coil structure connected in series to a proximal side of the distal end side region.

The structure insures a functional gradient characteristic to be flexible in front and rigid in rear along the lengthwise direction, thus enabling the user to a good maneuverability in the proximal side and a pliable follow-up capability in the distal end.

According to other aspect of the invention, the sheath body has a triple region structure which consists of a distal end side region having the deformed tight coil structure, an intermediary region having a wire-stranded hollow coil structure connected in series to a proximal side of the distal end side region, and a proximal end side region having a tubular structure connected in series to a proximal side of the intermediary region.

With the tubular structure provided in the proximal end side region, it is possible to readily transmit the rotational torque to the distal end side region, in addition to the advantages mention above.

### BRIEF DESCRIPTION OF THE DRAWINGS

Preferred forms of the present invention are illustrated in the accompanying drawings in which:

FIG. 1 is a side elevational view of a medical treatment equipment according to a first embodiment of the invention but partly sectioned;

FIG. 2 is a longitudinal cross sectional view of a main portion of the medical treatment equipment but partly broken;

FIG. 3 is a longitudinal cross sectional view of the main portion of the medical treatment equipment when bent;

FIG. 4 is a longitudinal cross sectional view of a prior medical treatment equipment when bent;

FIG. 5 is a schematic view showing how the prior medical treatment equipment is lengthened, as compared to the present invention;

FIG. 6 is a schematic view showing how a prior elongate core is lengthened with the increase of a total bending angle, as compared to the present invention;

FIG. 7 is a longitudinal cross sectional view of a main portion of a medical treatment equipment according to a second embodiment of the invention;

FIG. 8 is a longitudinal cross sectional view of the main portion of the medical treatment equipment when bent;

FIG. 9 is a longitudinal cross sectional view of a main portion of a medical treatment equipment according to a third embodiment of the invention;

FIG. 10 is a longitudinal cross sectional view of the main portion of the medical treatment equipment;

FIG. 11 is a longitudinal cross sectional view of a main portion of a medical treatment equipment according to a fourth embodiment of the invention;

FIG. 12 is a longitudinal cross sectional view of a main portion of a medical treatment equipment according to a fifth embodiment of the invention;

FIG. 13 is a side elevational view of a medical treatment equipment according to a sixth embodiment of the invention but partly sectioned;

FIG. 14 is a side elevational view of of a medical treatment equipment according to a seventh embodiment of the invention but partly sectioned;

FIG. 15 is a side elevational view of of a medical treatment equipment according to an eighth embodiment of the invention but partly sectioned;

FIG. 16 is an enlarged side elevational view showing how a clip works;

FIG. 17 is a longitudinal cross sectional view of a medical treatment equipment according to a ninth embodiment of the invention;

FIG. 18 is an enlarged longitudinal cross sectional view of a main portion of the medical treatment equipment;

FIG. 19 is an enlarged cross sectional view taken along lines XIX-XIX of FIG. 17;

FIG. 20 is a side elevational view of a medical treatment equipment according to a tenth embodiment of the invention but partly sectioned;

FIG. 21 is a side elevational view of a main portion of a medical treatment equipment according to an eleventh embodiment of the invention but partly sectioned; and

FIG. 22 is a side elevational view of a main portion of a medical treatment equipment according to a twelfth embodiment of the invention but partly sectioned.

### DETAILED DESCRIPTION OF THE PREFERRED EMBODIMENTS

In the following description of the depicted embodiments, the same reference numerals are used for features of the same type. A distal end portion designates a left side, and a proximal end portion designates a right side all through the drawings of the invention.

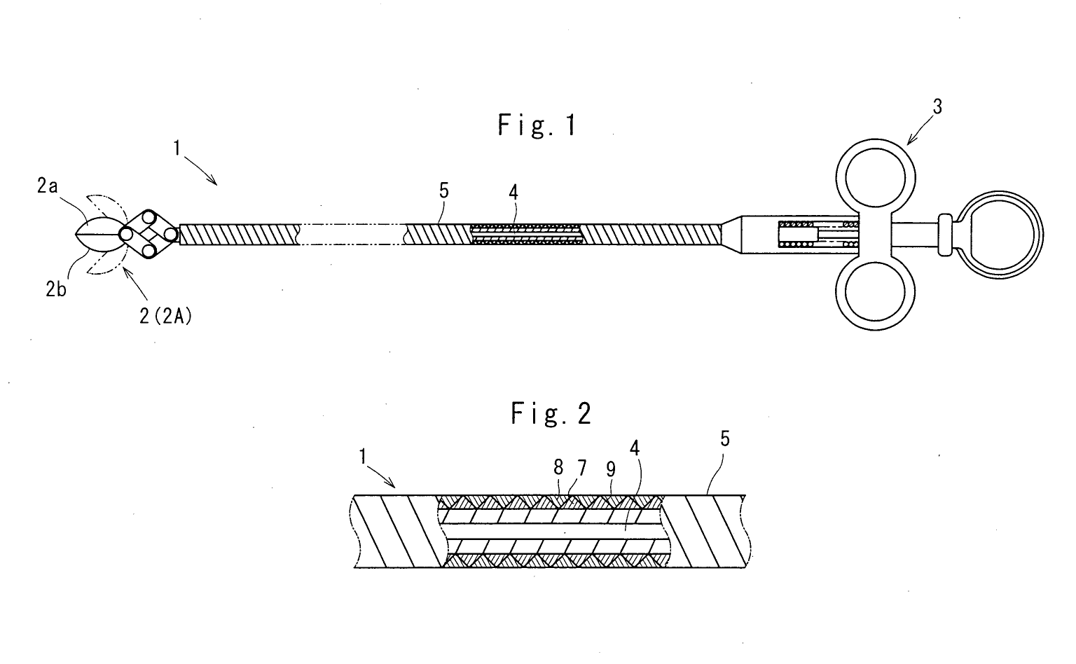

Referring to FIGS. 1 through 6 which show a medical treatment equipment 1 according to a first embodiment of the invention, a distal end portion of the medical treatment equipment 1 has a treatment portion 2, and a proximal end portion of the medical treatment equipment 1 has an operational portion 3. Between the treatment portion 2 and the operational portion 3, there is provided an elongate core 4 which connects the operational portion 3 to the treatment portion 2. A long flexible sheath body 5 is provided to have the elongate core 4 inserted therethrough as shown in FIGS. 1 and 2.

The medical treatment equipment 1 works as an endoscopic treatment device in which the treatment portion 2 is a biopsy cup 2A provided to open and shut its blades 2a, 2b of scissors by a push-pull manipulation of the elongate core 4 through the operational portion 3. Through the push-pull manipulation of the elongate core 4 by way of the operational portion 3, the elongate core 4 axially moves within the sheath body 5 to open and shut the biopsy cup 2A which is connected to a distal end of the elongate core 4 and that of the sheath body 5.

In the medical treatment equipment 1, the sheath body 5 has a deformed tight coil structure in which a plurality of wire elements 7, 8 (two wires in the present embodiment of the invention) are formed substantially triangular in cross section, and helically wound with one coil line turn alternately arranged in neighborhood of other coil line turn.

An apex of the triangular cross section of the one coil line turn faces an inner diameter side of the sheath body 5, and an apex of the triangular cross section of the other coil line turn faces an outer diameter side of the sheath body 5.

One lateral side of the triangular cross section of the one coil line turn and one lateral side of the triangular cross section of the other coil line turn come in contact with each other to form a mutual neighborhood side 9.

Lateral sides of neighboring triangular cross sections are relatively slidable each other at the mutual neighborhood side 9 in a radial direction of the sheath body 5 when the sheath body 5 is bent. The sheath body 5 forms the deformed tight coil structure through an entire length of the sheath body 5. The cross sectional shapes of the wire elements 7, 8 are identical each other to form a right-angled isosceles triangle. The apex of the triangular cross section of the wire element 7 faces the inner diameter side of the sheath body 5, and the apex of the triangular cross section of the wire element 8 faces the outer diameter side of the sheath body 5.

It is to be noted that the wire elements 7, 8 may be combined beforehand to be helically wound upon providing the deformed tight coil structure. Otherwise, the wire element 8 may be helically wound over the wire element 7 so that the apex of the triangular cross section of the wire element 8 faces the inner diameter side of the sheath body 5, after the wire element 7 helically wound with the apex of the triangular cross section faced the outer diameter side of the sheath body 5 (so as to interpose the apex portion of the wire element 8 between the coil line turns (valley portion) of the wire element 7).

An actuation and advantages of the sheath body 5 in the first embodiment of the invention are explained as follows:

FIG. 4 shows a longitudinal cross sectional view of a prior sheath body S when the sheath body S is bent in which a single wire (circular in cross section) is helically wound to form a tight knit coil structure.

Due to the tight knit coil structure, a bending force causes to develop a line gap C between the coil line turns of a wire element W at an outer side of the neutral line (central line) of the sheath body S, the gap C of which is proportional to a tensile force applied to the sheath body S when the sheath body S is subjected to the bending force along a bent portion of the somatic cavity. Namely, the bending force curvedly deforms the sheath body S with the straight-oriented restitutive force remained within the sheath body S.

The straight-oriented restitutive force urges the sheath body S to strongly press both ends of the curvedly deformed portion against a vascular wall or the like, and subsequently the sheath body S slides or sets the curvedly deformed portion with the curvedly deformed portion pressed against the vascular wall or the like. This will do a harm to the vascular wall, the somatic wall or the like, and obstruct a good insertability upon advancing the sheath body S into the somatic cavity, a procedure of which is to be followed.

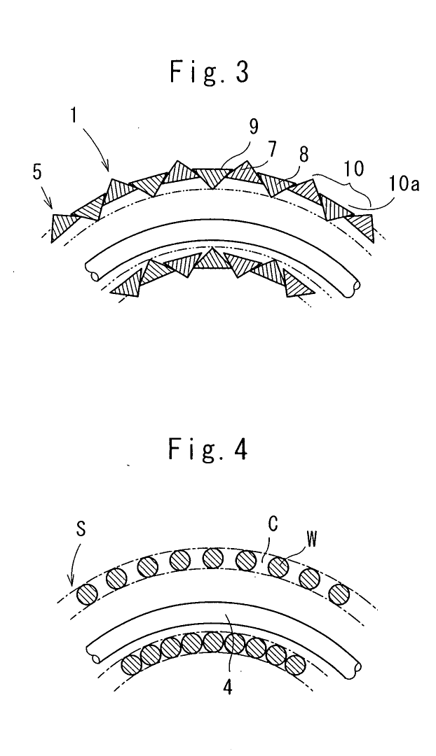

In the meanwhile, FIG. 3 shows an explanatory view of the medical treatment equipment 1 when it is curvedly deformed.

The sheath body 5 of the medical treatment equipment 1 has the deformed tight coil structure in which the plurality of wire elements 7, 8 are formed substantially triangular in cross section, and helically wound with one coil line turn alternately arranged in neighborhood of other coil line turn. The apex of the triangular cross section of the one coil line turn faces the inner diameter side of the sheath body 5, and the apex of the triangular cross section of the other coil line turn faces the outer diameter side of the sheath body 5. One lateral side of the triangular cross section of the one coil line turn and one lateral side of the triangular cross section of the other coil line turn are in contact with each other to form a mutual neighborhood side 9. The lateral sides of neighboring triangular cross sections are relatively slidable each other at the mutual neighborhood side 9 in the radial direction of the sheath body 5 when the sheath body 5 is curvedly bent.

In more specific, the lateral side of the triangular cross section of the wire element 7 slides against the neighboring lateral side of the triangular cross section of the wire element 8 at the mutual neighborhood side 9. One lateral side slides toward the central line of the sheath body 5, and other lateral side slides away from the central line of the sheath body 5. The sliding action of the lateral sides of the wire elements 7, 8 absorbs both the tensile and contractile stresses developed when the sheath body 5 is curvedly bent.

For this reason, it is possible to readily bend the sheath body 5 without inviting the line gap between the coil line turns of the wire elements 7, 8. In more tangible terms, it is possible to curvedly deform the sheath body 5 freely depending on the curved configuration of the vascular wall without residing the bending stresses inside the sheath body 5, thus enabling the user to easily change the sheath body 5 from the bend-shaped configuration to the straight-shaped configuration and vice versa, so as to attain a remarkable plasticity between the straight-shaped configuration and the bend-shaped configuration.

The plasticity means to enable the user to freely change the sheath body 5 into the bend-shaped configuration and the straight-shaped configuration while maintaining each of the configuration upon displacing the sheath body 5 between the bend-shaped configuration and the straight-shaped configuration.

The sheath body 5 develops substantially no straight-oriented restitution force in the sheath body 5 due to the bending deformation after setting the sheath body 5 in the somatic cavity or while inserting the sheath body 5 into the somatic cavity. The sheath body 5 obviates the fear of doing harm to the vascular wall or the like when bent in the somatic cavity, thus enabling the user to a good insertability toward the somatic cavity when advancing the sheath body 5 into the somatic cavity, a procedure of which is to be subsequent, so as to overcome the inconveniences involving the prior sheath body S.

Due to the sheath body 5 which is bendable without inviting the line gap between the coil line turns of the wire elements 7, 8, it is possible to prevent the sheath body 5 from being lengthened when curvedly bent.

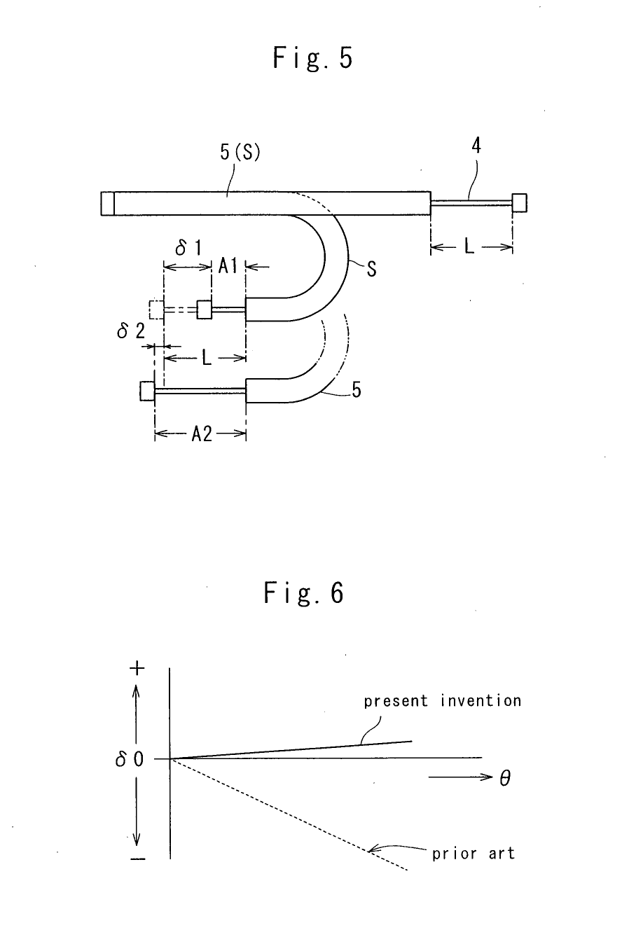

In order to explain why the above advantage is obtained, a comparative model is presented in which a distal end of the elongate core 4 is secured to each distal end of the prior sheath body S and the sheath body 5 as shown in FIGS. 5 and 6. The prior sheath body S and the sheath body 5 have the same lengthwise dimension, and the elongate core 4 has a same protracted length L each beyond a proximal end of the prior sheath body S and the sheath body 5.

Upon bending the prior sheath body S, the sheath body S retracts the elongated core 4, and renders a protracted length A1 shorter than the initial length L before bent into the bend-shaped configuration, so as to make a displacement δ 1 negative between the protracted length A1 and the length L (δ 1=A1−L).

On the other hand, the sheath body 5 renders a protracted length A2 somewhat longer than the initial length L before bent into the bend-shaped configuration, so as to make a displacement δ 2 positive between the protracted length A2 and the length L (δ 2=A2−L)

The reason why the sheath body S renders the protracted length A1 shorter when bent, is that the sheath body S develops the line gap C to lengthen its entire length so as to retract the elongate core 4 into the sheath body S to such a degree as the sheath body S is lengthened when curvedly bent (refer to FIG. 4).

The reason why the sheath body 5 renders the protracted length A2 somewhat longer when bent, is that the sheath body 5 develops no line gap C between the coil line turns of the wire elements 7, 8 due to the sliding action therebetween, thus remaining the entire length substantially unchanged, and residing the elongate core 4 along the inner side (the shortest curved side distance) of the sheath body 5 when curvedly bent (refer to FIG. 3).

FIG. 6 shows a relationship between a total bending angle (θ) and the displacement (δ) of the elongate core 4 when curvedly bent. In FIG. 6, the sheath body 5 is represented by solid line and the sheath body S represented by broken lines for the purpose of comparison. The sheath body S gradually decreases the protracted length L with the increase of the total bending angle (θ).

Upon bending the sheath body S in the somatic cavity while advancing it along a curved tract, the sheath body S lengthens its entire length, and exceedingly reduces an effective action length of the elongate core 4 to develop unnecessary harmful tension on the elongate core 4, thus functionally deteriorating the treatment portion 2 so as to lose a good therapeutic implementation of the diseased area.

Contrary to the prior sheath body S, the sheath body 5 remains the protracted length L substantially unchanged even with the increase of the total bending angle (θ).

Upon bending the sheath body S several times in the somatic cavity, the sheath body 5 neither lengthens its entire length nor reduces an effective action length of the elongate core 4 to develop no unnecessary harmful tension on the elongate core 4, thus functionally maintain the treatment portion 2 so as to stably carry out a good therapeutic implementation of the diseased area.

Moreover, actuations and advantages characteristic of the sheath body 5 are obtained according to the first embodiment of the invention. In the deformed tight coil structure of the sheath body 5, the neighboring wire elements 7, 8 alternately slide toward the central line of the sheath body 5 and away from the central line of the sheath body 5 in the mutual neighborhood side 9, so as to develop an undulation (concave-convex portion 10) on an outer surface of the sheath body 5 (refer to FIG. 3). A concave portion 10a of the concave-convex portion 10 serves as an echo-reflective area which is necessary to insure a clear supersonic image on the monitor under a three-dimensional supersonic echo.

In order to generally insure the supersonic image clearly on the monitor under the three-dimensional supersonic echo, it is desirable to provide a concave portion as the echo-reflective area. This is because the concave portion tends to readily reflect the supersound compared to the cellular tissue structure. Because of the exceedingly large difference of acoustic characteristic impedance at a boundary between the cellular tissue structure and the concave portion, most of the supersound are reflected at the boundary between the cellular tissue structure and the concave portion. The acoustic characteristic impedance is represented by the product (ρ c) of sound velocity (c) and the textural density (ρ) of the cellular tissue structure.

The concave-convex portion 10 obviates to provide the concave portion (e.g., groove) on the outer surface of the sheath body 5 because the sheath body 5 develops the concave-convex portion 10, the concave portion 10a of which serves as the echo-reflective area when the sheath body 5 is curvedly bent. With the concave portion 10a shaped in the form of a consecutive spiral groove, it is possible to visually recognize the image as the continuously curved configuration.

With the cross sectional shapes (right-angled isosceles triangle) of the wire elements 7, 8 are provided to be identical each other, it is sufficient to produce a single type of common wire elements, thus improving the productivity with the manufacturing cost reduced. With the cross sectional shapes of the wire elements 7, 8 having the equilateral oblique sides, and angles of the equilateral oblique sides having 45 degrees, it is possible to equipoise a dual component force along the equilateral oblique sides, thereby enabling the user to smoothly slide the neighboring wire elements 7, 8 in the radial direction when the sheath body is deformed from the straight-shaped configuration to the bend-shaped configuration and vice verse.

Actuations and advantages are explained when the medical treatment equipment 1 is used as the endoscopic treatment device for collecting the cellular tissue structure. As the treatment portion 2, the medical treatment equipment 1 uses the biopsy cup 2A which is actuated to open and shut by the push-pull manipulation of the operational portion 3.

By pushing the operational portion 3 to retract the elongate core 4 into the sheath body 5, it is possible to open the biopsy cup 2A to collect the cellular tissue structure as shown at the phantom lines in FIG. 1. By pulling the operational portion 3 to withdraw the elongate core 4 from the sheath body 5, it is possible to shut the biopsy cup 2A with the cellular tissue structure interposed between the blades 2a, 2b of the scissors. It is to be noted that the pulling action may be manually done or done by using a return spring (not shown).

When the prior sheath body S is employed to the medical treatment equipment, the sheath body S lengthens its entire length upon curvedly bending sheath body S. For this reason, the sheath body S is subjected to the bending force, and abruptly deforms along the shape of the curved area in the somatic cavity while advancing the sheath body S through the somatic cavity. This cause to unexpectedly open, shut or rotate the biopsy cup so as to injure an inner wall of the somatic cavity.

Contrary to the prior sheath body S, with the sheath body 5 prevented from being lengthened upon bending the sheath body 5 in the somatic cavity, it is possible to actuate the biopsy cup 2A to open and shut in the stable fashion upon collecting the cellular tissue structure even when the sheath body 5 accumulates a greater amount of bends before reaching the biopsy cup 2A to a target destination in the somatic cavity.

Namely, although the bending operation causes to curvedly bend the sheath body 5, the bending operation causes no shortage of an operational stroke (no operational shortage of the elongate core 4), thereby enabling the user to insure that the biopsy cup 2A can quickly responds to the open and shut action of the operational portion 3.

With the concave portion 10a provided on the sheath body 5 as the echo-reflective area, it is possible to clearly delineate the supersonic image under the three-dimensional supersonic echo. This enables the user to extremely easily collect the cellular tissue structure in the somatic cavity as a way of therapeutically treating the diseased portion.

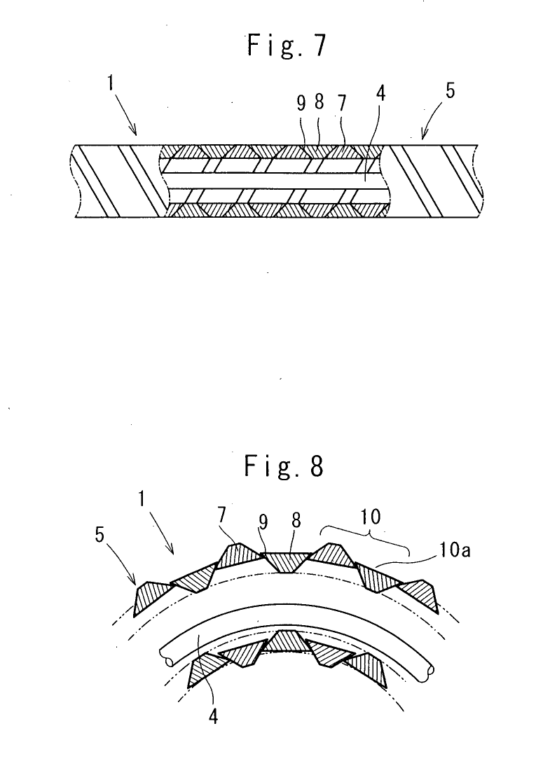

FIGS. 7 and 8 show a second embodiment of the invention in which the identical cross sectional shapes of the wire elements 7, 8 are equilateral trapezoids with the oblique sides forming 45 degrees against a lower long side.

Among the cross sectional shapes of the wire elements 7, 8, the wire element 7 makes an upper short side (except for the oblique lateral sides) of the trapezoid face an outer diameter side of the sheath body 5, and the wire element 8 makes the upper short side of the trapezoid face an inner diameter side of the sheath body 5 as shown in FIG. 7.

In the second embodiment of the invention, the neighboring wire elements 7, 8 make the lateral sides of the trapezoids slide each other at the mutual neighborhood side 9 upon bending the sheath body 5 as shown in FIG. 8.

The sliding movement between the lateral sides of the trapezoids absorbs both the tensile stresses and the contractile stresses developed on the sheath body 5 due to the bending deformation, thus resultantly preventing the sheath body 5 from being lengthened. The sheath body 5 develops the convex-concave portion 10 on the outer surface of the sheath body 5 when curvedly bent, so as to attain the same advantages as mentioned in the first embodiment of the invention.

The same advantages are achieved as the first embodiment of the invention by defining the identical cross sectional shape. The same advantages are derived as the first embodiment of the invention by defining the trapezoid to have the equilateral oblique sides with the lateral sides forming 45 degrees against the lower long side of the trapezoid.

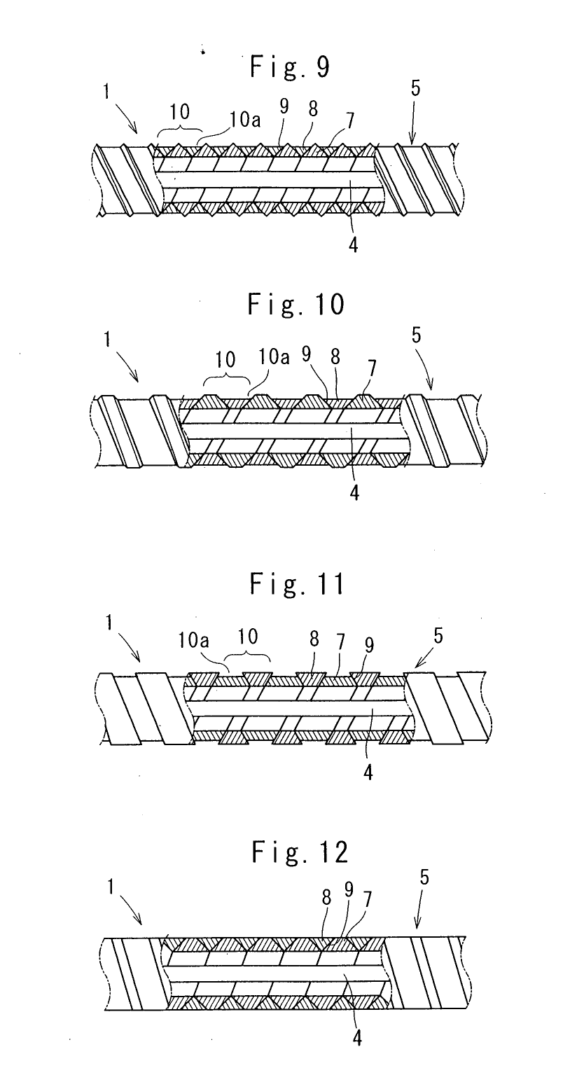

FIGS. 9 and 10 show a third embodiment of the invention in which the cross sectional shapes of the wire elements 7, 8 are analogous each other so that either one of the wire elements 7, 8 is smaller in scale than the other.

With the wire elements 7, 8 defined to be at least analogous each other, it is possible for the wire elements 7, 8 to form the tight knit coil structure even if the cross sectional shapes of the wire elements 7, 8 are not necessarily the same. Such is the structure that the sheath body 5 forms the concave-convex portion 10 (concave portion 10a) even in the straight-shaped configuration. This enables the user to clearly delineate the supersonic image under the three-dimensional supersonic echo.

FIG. 11 shows a fourth embodiment of the invention in which the wire elements 7, 8 are categorically trapezoid in cross section. The cross sectional shape of the wire elements 7 has the same gradient angle of the oblique side as that of the wire element 8 but having a breadth and height different than that of the wire element 8.

In the fourth embodiment of the invention, it is possible to to clearly delineate the supersonic image under the three-dimensional supersonic echo.

FIG. 12 shows a fifth embodiment of the invention in which the wire elements 7 is generally trapezoidal in cross section, and the wire elements 8 is generally triangular in cross section. The upper short side of the trapezoidal cross section of the wire elements 7 faces the outer diameter side of the sheath body 5, and the apex of the triangular section of the wire elements 8 faces the inner diameter side of the sheath body 5. Conversely, the upper short side of the trapezoidal cross section of the wire elements 7 may face the inner diameter side of the sheath body 5, and the apex of the triangular section of the wire elements 8 may face the outer diameter side of the sheath body 5.

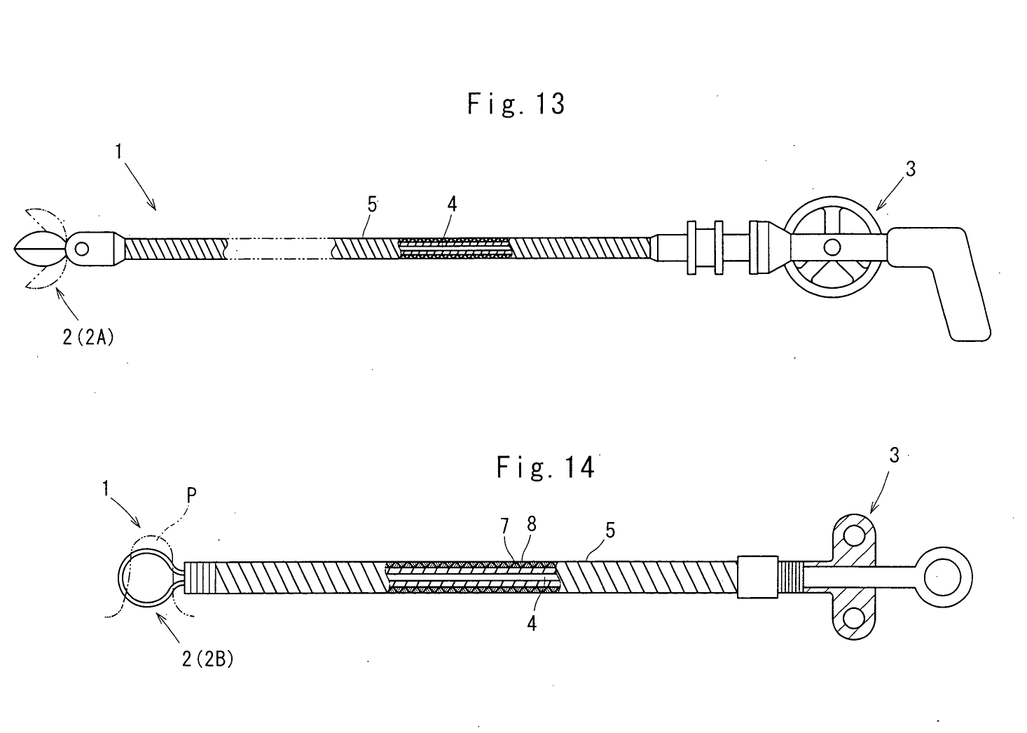

FIG. 13 shows a sixth embodiment of the invention in which the medical treatment equipment 1 applies the treatment portion 2 to the biopsy cup 2A (biopsy forceps for endoscope) which is actuated to open and shut by the push-pull manipulation of the operational portion 3.

In the sixth embodiment of the invention (endoscopic treatment device), the same advantages are attained as achieved by the first embodiment of the invention.

FIG. 14 shows a seventh embodiment of the invention in which the medical treatment equipment 1 applies the treatment portion 2 to a looped wire 2B for tightly tying up the cellular tissue structure in the somatic cavity. During the process in which medical treatment equipment 1 is operated, by pulling the elongate core 4 through the operational portion 3, it is possible to diametrically reduce the looped wire 2B so as to form the endoscopic treatment structure. The endoscopic treatment structure enables the user to diametrically reduce the looped wire 2B through the push-pull and rotational manipulation of the operational portion 3, so as to tightly tie up the cellular tissue structure (e.g., polyp P) in the somatic cavity.

When the prior sheath body S is employed to the medical treatment equipment 1, the sheath body S is subjected to the bending force, and abruptly deforms staunchly along the shape of the curved area of the somatic cavity during the process in which the sheath body S is inserted through the somatic cavity. This retracts the elongate core 4 into the sheath body 5, and inconveniently tightens the looped wire 2B.

When the sheath body S is bent several times and accumulating a greater amount of bends on the sheath body S during the process in which the sheath body S is inserted, the sheath body S is more lengthened to develop the tensile force on the elongate core 4 until the treatment portion 2 reaches the target destination. The tensile force deteriorates the maneuverability of the looped wire 2B upon binding and loosening the looped wire 2B, thus getting the looped wire 2B come out of the polyp P due to the shortage of the binding force, or inconveniently getting the polyp P bound excessively tight.

Contrary to the prior sheath body S, the sheath body 5 has the deformed tight coil structure, and having the advantages as mentioned in the first embodiment of the invention. For this reason, even if the sheath body 5 is bent several times and accumulating a greater amount of bends on the sheath body 5 before reaching the target destination in the somatic cavity, it is possible to diametrically reduce and increase the looped wire 2B in the stable fashion through the operational portion 3 upon collecting the cellular tissue structure in the somatic cavity.

Namely, the sheath body 5 causes no shortage of the operational stroke (operational shortage of elongate core) due to the bend-shaped configuration even when the sheath body 5 is curvedly bent in the somatic cavity. This makes it possible to quickly actuate the looped wire 2B in response to the operational portion 3, and maintain a good function to bind the cellular tissue structure tight.

With the concave portion 10a (echo-reflective area) developed on the sheath body 5 upon curvedly bending the sheath body 5, it is possible to clearly delineate the supersonic image under the three-dimensional supersonic echo. This enables the user to extremely easily collect the cellular tissue structure in the somatic cavity as a way of therapeutically treating the diseased area.

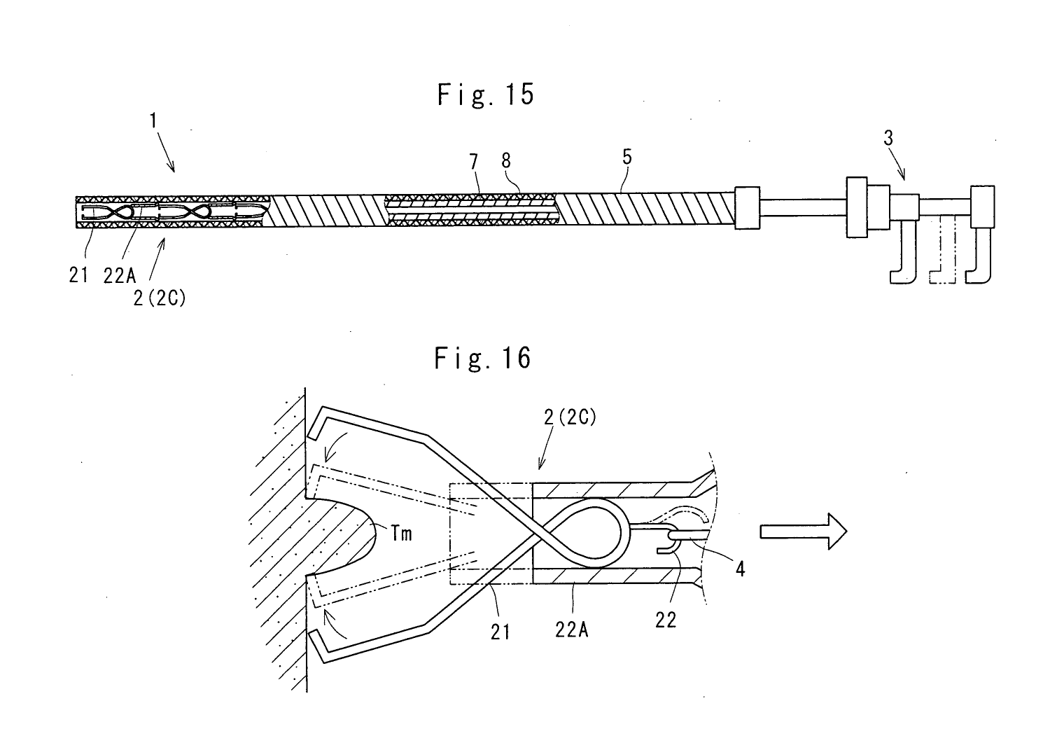

FIGS. 15 and 16 show an eighth embodiment of the invention in which the medical treatment equipment 1 accommodates a clip 21 within the sheath body 5, and applies the treatment portion 2 to a clip retainer 2C which retains the clip 21 in the cellular tissue structure in the somatic cavity by the push-pull manipulation of the operational portion 3 through the elongate core 4, so as to form a multi-functional surgical treatment structure.

During the process in which the medical treatment equipment 1 is operated, the elongate core 4 hooks its distal end to a clip hook portion 22 of the clip 21 by means of the operational portion 3. The clip 21 is accommodated into a clip ring 22A when placed within the sheath body 5. At the time of pushing the clip ring 22A out the sheath body 5, the pull operation makes the elongate core 4 move with its reaction force supported by the sheath body 5 (deformed tight coil structure), so as to expand the clip hook portion 22 to release the clip 21 as shown at the phantom lines in FIG. 16, thus enabling the user to retain the clip 21 in the somatic cavity with the cellular tissue structure Tm held by the clip 21.

When the prior sheath body S is employed to the medical treatment equipment 1, the sheath body S is subjected to the bending force, and develops the line gap C (refer to FIG. 3) at the tensile side of the coil line turns of the wire elements 7, 8, thus permitting the clip 21 to invade at the proximal side of the sheath body S to bring the clip 21 a positional shift from the distal end of the sheath body S.

Upon subjecting the sheath body S to the contractile reaction through the operation of the proximal side so as to decrease the line gap C of the coil line turns of the wire elements 7, 8, thus permitting the sheath body S to oscillate so as to injure the digestive tract or the like depending on the operational force given to the sheath body S.

When the sheath body S accumulates a greater amount of bends on the sheath body S until the treatment portion 2 reaches the target destination, the sheath body S is lengthened to a greater degree so as to make it difficult to transmit the predetermined tensile stroke to the treatment portion 2, thus inviting a functional defect defying to expand the clip hook 22 necessary to retain in the somatic cavity.

Due to the deformed tight coil structure according to the eighth embodiment of the invention, the medical treatment equipment 1 has the same actutions and advantages as mentioned in the first embodiment of the invention. This prevents the positional shift of the clip 21 within the sheath body 5 upon curvedly bending the sheath body 5, thus bringing no shortage of the operational stroke for the elongate core 4 so as to insure a quick response toward the clip-retaining action to perform a stable clip-retaining function.

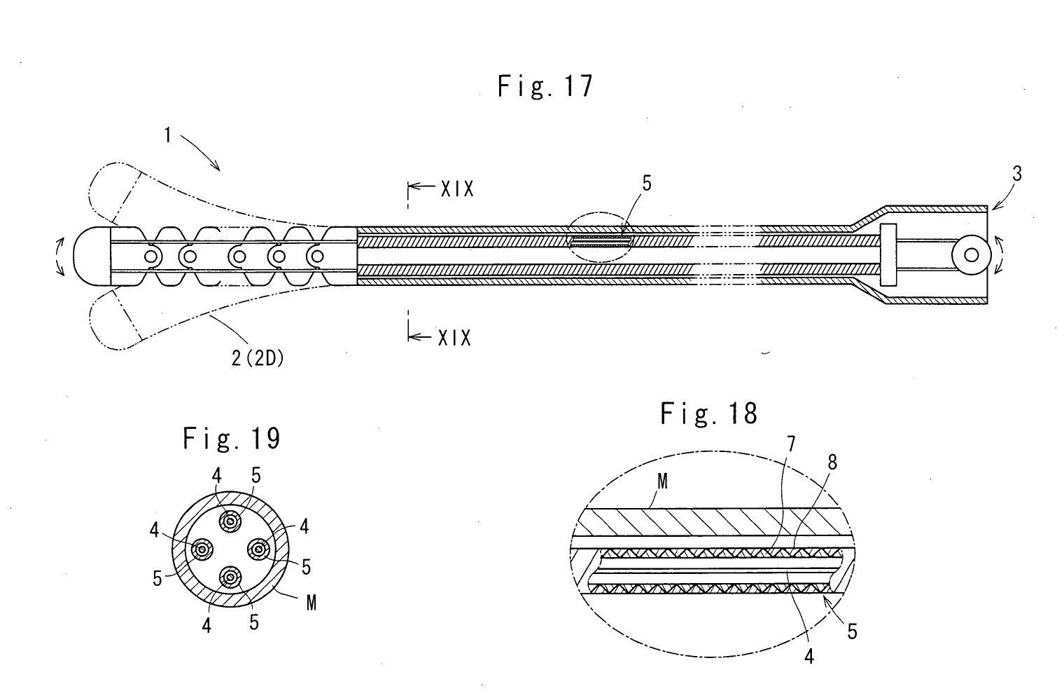

FIGS. 17, 18 and 19 show a ninth embodiment of the invention in which the medical treatment equipment 1 applies the treatment portion 2 to an endoscope-angle shifting portion 2D (endoscopic structure) which is altered by adjusting the tension of the elongate core 4 through the rotational movement of the operational portion 3.

A plurality of sheath bodies 5 (hypotubes) are provided between the operational portion 3 and the treatment portion 2 within an elongate tube M as shown in FIGS. 17, 18.

By way of illustration, four sheath bodies 5 are arranged along the circumferential direction at regular intervals as shown in FIG. 19.

During the process in which the medical treatment equipment 1 is operated, the rotational movement of the operational portion 3 adjusts the tension of the elongate core 4 to change the endoscope-angle shifting portion 2D to orient an endoscope toward the desired direction.

When the prior sheath body S is employed to the medical treatment equipment, the sheath body S is subjected to the bending force only along the shape of the somatic cavity, and unexpectedly alters the angle of the endoscope during process in which the sheath body S is advanced in the somatic cavity.

Due to the deformed tight coil structure according to the ninth embodiment of the invention, the medical treatment equipment 1 has the same actuations and advantages as mentioned in the first embodiment of the invention. This prevents the unexpected alteration of the angle of the endoscope upon curvedly bending the sheath body 5, thus bringing no shortage of the operational stroke for the elongate core 4 so as to insure a quick response toward the shiftable action of the angle of the endoscope.

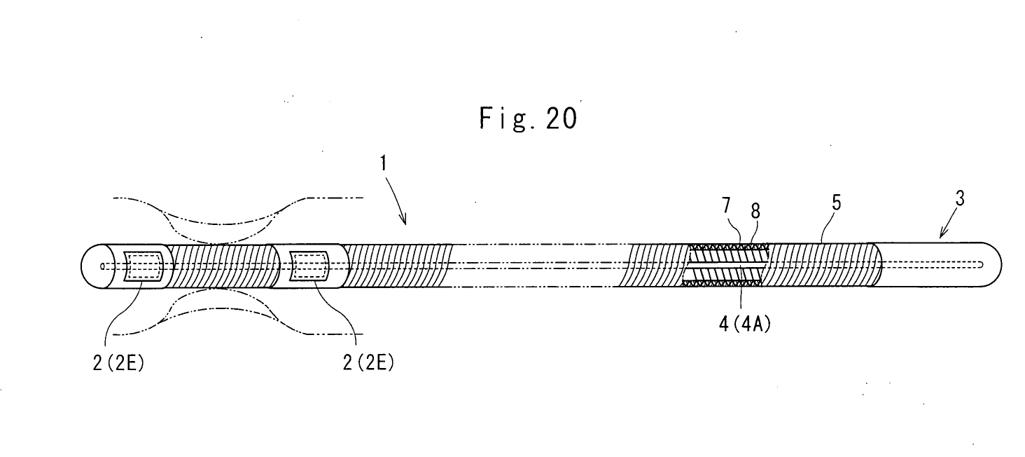

FIG. 20 shows a tenth embodiment of the invention in which the medical treatment equipment 1 has a sensor 2E built in the sheath body 5 to form a sensor-attached guide wire structure.

In more specific, the medical treatment equipment 1 provides a lead wire 4A instead of the elongate core 4, and connects the sensor 2E to a distal end of the lead wire 4A as the treatment portion 2. During the process in which the medical treatment equipment 1 is operated, the sheath body 5 is inserted into the somatic cavity with the operational portion 3 held by hand, so that the sensor 2E can measure the blood pressure or monitor the blood pressure wave.

When the prior sheath body S is employed to the medical treatment equipment, and especially inserted into the blood vessel to confirm the post-operational condition of the coronary occlusion, the sheath body S is subjected to the bending force at the bent portion, and lengthened to stretch the lead wire 4A enough to permit a disconnection so as to lose the normal function of the sensor 2E. At the abruptly bent portion, the sheath body S develops the line gap C between the coil line turns of the wire elements 7, 8 to induce a turbulance in the blood vessel. The turbulance renders the blood pressure waves unstable to obstruct the normal measurement of the blood pressure waves.

Due to the deformed tight coil structure according to the tenth embodiment of the invention, the medical treatment equipment 1 has the same actuations and advantages as mentioned in the first embodiment of the invention. It is possible to protect the sensor 2E against the turbulance in the blood vessel which otherwise would be developed due to the line gap appeared between the coil line turns, so as to render the output waves unstable when the sensor 2E generates the output waves. With the sheath body 5 prevented from being lengthened, it is possible to protect the lead wire 4A against the disconnection so as to insure the continued monitoring.

FIG. 20 shows an eleventh embodiment of the invention in which the sheath body 5 has a dual region structure which consists of a distal end side region 5a having the deformed tight coil structure and a proximal end side region 5b having a wire-stranded hollow coil structure connected in series to a proximal side of the distal end side region 5a.

The structure insures a functional gradient characteristic to be flexible in front and rigid in rear along the lengthwise direction, that is to say, rendering the distal end side region 5a to be flexible in the deformed tight coil structure, and resultantly forming a wire-stranded hollow coil structure in which the proximal end side region 5b is more rigid that the distal end side region 5a.

Since the medical treatment equipment 1 inserts the sheath body 5 deeply into the curved thin tract (somatic cavity) such as the digestive tract, the sinuous path of the blood vessel or the like, it is preferable for the sheath body 5 to attain a good pushability and good rotational maneuverability with the rigidity in rear, and achieving a smooth, deep insertability with the flexibility in front, due to the functional gradient characteristic to be flexible in front and rigid in rear along the lengthwise direction.

According to the eleventh embodiment of the invention, it is possible to perform the actuations and the advantages uniquely derived from the deformed tight coil structure as mentioned in the first embodiment of the invention.

Due to the wire-stranded hollow coil structure, it is possible to enhance the torque transmissibility from the proximal end side to the distal end side, contrary to a single helical coil wire structure.

Since the single helical coil wire structure supports the rotational torque along the single wire element, when the single helical coil wire structure comes contact with the curved inner wall in the somatic cavity, the single helical coil wire structure forms different turns of coil lines depending on the contact conditions and positions against the curved inner wall upon rotating the operational portion at the proximal side. This hampers a good torque transmissibility from the proximal end side to the distal end side.

Contrary to the single helical coil wire structure, the wire-stranded hollow coil structure has the plurality of the wire elements 7, 8, coil turns of which incline against the central line of the wire-stranded hollow coil structure, so as form a gradient angle which is greater than that of the single helical coil wire structure. This enables the user to smoothly transmit the rotational torque from the proximal end side to the distal end side.

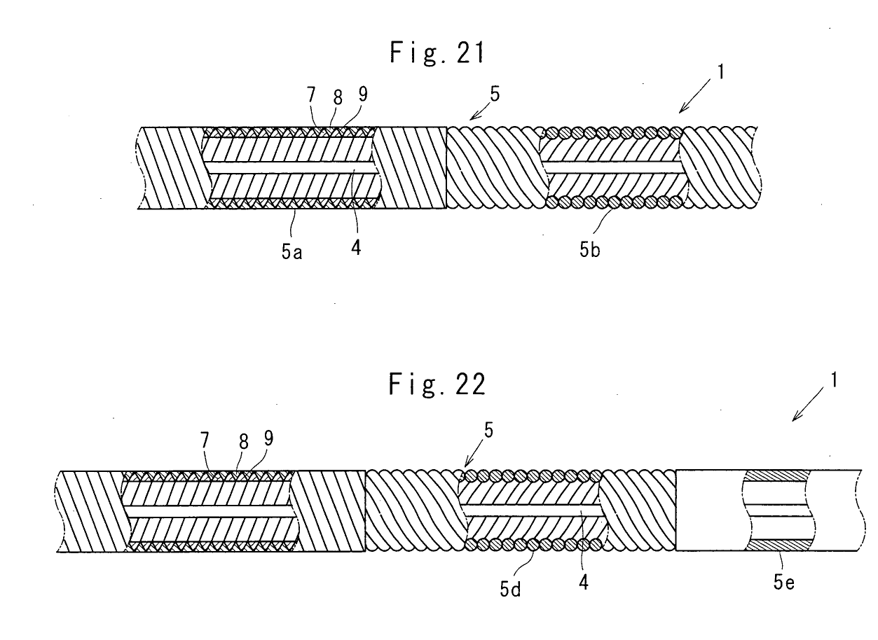

FIG. 21 shows a twelfth embodiment of the invention in which the sheath body 5 has a triple region structure which consists of a distal end side region 5c having the deformed tight coil structure, an intermediary region 5d having a wire-stranded hollow coil structure.

The intermediary region 5d of the wire-stranded hollow coil structure is connected in series to a proximal side of the distal end side region 5c. A proximal end side region 5e is provided to have a tubular structure connected in series to a proximal side of the intermediary region 5d.

The structure insures a functional gradient characteristic to be flexible in front and rigid in rear along the lengthwise direction, in addition to the actuations and the advantages uniquely derived from the deformed tight coil structure as mentioned in the first embodiment of the invention.

With the tubular structure provided in the proximal end side region 5e, it is possible to readily transmit the rotational torque to the distal end side region 5c.

To supplementarily explain the situation, it is preferable to form the wire-stranded hollow coil structure into a rope-like configuration by twisting a plurality of wire elements with the use of a rope-twisting machine after withdrawing a central core outside. Otherwise, the plurality of wire elements may be helically stranded into a hollow coil structure.

At the time of stranding the plurality of wire elements, these wire elements may be forced through a wave-shaping die or a preforming die, so as to circumferentially provide a transformation layer (work-hardening layer) on an entire surface of the wire-stranded hollow coil structure, so as to be structurally homogenized in the full lengthwise direction.

In the case in which a multitude of wire elements are wound around a mandrel, the case is liable to produce a line gap each time when the wire elements are wound around the mandrel.

The connection among the distal end side region 5c, the intermediary region 5d and the proximal end side region 5e may be done by means of a welding procedure, a soldering procedure, otherwise through a tubular-piece joint or the like.

As one example of the wire-stranded hollow coil structure, twelve stainless steel wires (austenitic stainless steel wires, 0.33 mm in line diameter) may be stranded to form the wire-stranded hollow coil structure which measures 1.6 mm in outer diameter. Instead of the twelve stainless steel wires, seven stainless steel wires may be used with the line diameter as 0.11 mm instead of 0.33 mm.

Upon forming the wire-stranded hollow coil structure, either stainless steel wires or superelastic metal wires (Ni—Ti alloy wires) may be used, otherwise the stainless steel wires and the superelastic metal wires may be united.

Any of the stainless steel wires or the Ni—Ti alloy wires may be applied to the tubular structure of the proximal end side region 5e.

In the case in which the cross sectional shape of the wire elements 7, 8 is the equilateral triangle upon forming the deformed tight coil structure, dimensions of the base side and the height measure 0.35 mm and 0.30 mm respectively with an outer diameter of the deformed tight coil structure as 1.6 mm.

In the case in which the cross sectional shape of the wire elements 7, 8 is the equilateral trapezoid, dimensions of the upper short side, the lower long side and the height measure 0.35 mm, 0.70 mm and 0.30 mm respectively with the outer diameter of the deformed tight coil structure as 1.6 mm, otherwise the dimensions of the upper short side, the lower long side and the height in turn measure 0.30 mm, 0.90 mm and 0.30 mm with the equilateral side forming 45 degrees against the lower long side.

In order to make the coil-forming procedure easy, one of the wire elements 7, 8 of the stainless steel may be heat treated to be rendered into a non-rigid material (tensile strength: 80-120 kg/mm²), and combined to the other wire (tensile strength: hardened to be 180-300 kg/mm²) of the wire elements 7, 8.

Namely, one of the wire elements 7, 8 may be annealed by the heat treatment. The heat treatment may be carried out at the temperature of 600-900° C. for about 15-120 minutes. The conditions of the heat treatment vary depending on the line diameter and the material used for the wire elements 7, 8. By making the tensile strength and the hardness (mechanical strength) different between the wire elements 7, 8, it is possible to make the strength-lower wire unite the strength-higher wire so as to render the wire-winding procedure easy. The Ni—Ti alloy wire (shape-memory alloy wire) may be used to one of the wire elements 7, 8.

It is to be noted that upon forming the deformed tight coil structure from the first to the twelfth embodiment of the invention, three or more wire elements may be used instead of the two wire elements 7, 8.

It is also to be noted that the cross sectional shape of the wire elements 7, 8 may have oblique sides of different angle, instead of the equilateral triangle and the equilateral trapezoid defined from the first to the twelfth embodiment of the invention.

It is to be appreciated that a synthetic resin layer may be coated on the outer surface of the sheath body 5 to form a space surrounded by the synthetic resin layer and the wire elements 7, 8 when curvedly bent, so as to make the space function as the echo-reflective area. This makes it possible to more clearly delineate the supersonic image under the three-dimensional supersonic echo.

What is claimed is:

1. In a medical treatment equipment which includes a treatment portion provided at a distal end portion, an operational portion provided at a proximal portion, an elongate core provided to connect said treatment portion to said operational portion, and an elongated flexible sheath body, to which said elongate core is inserted;
said medical treatment equipment comprising:
said sheath body having a deformed tight coil structure in which a plurality of wire elements are formed triangular in cross section, and helically wound with one coil line turn alternately arranged in neighborhood of other coil line turn;
an apex of said triangular cross section of said one coil line turn facing an inner diameter side of said sheath body, and an apex of said triangular cross section of said other coil line turn facing an outer diameter side of said sheath body;
one lateral side of said triangular cross section of said one coil line turn and one lateral side of said triangular cross section of said other coil line turn being in contact with each other to form a mutual neighborhood side; and
lateral sides of neighboring triangular cross sections being relatively slidable each other at said mutual neighborhood side in a radial direction of said sheath body when said sheath body is bent.

2. In a medical treatment equipment which includes a treatment portion provided at a distal end portion, an operational portion provided at a proximal portion, an elongate core provided to connect said treatment portion to said operational portion, and an elongated flexible sheath body, to which said elongate core is inserted;
said medical treatment equipment comprising:
said sheath body having a deformed tight coil structure in which a plurality of wire elements are formed trapezoidal in cross section, and helically wound with one coil line turn alternately arranged in neighborhood of other coil line turn;
an upper short side of said trapezoidal cross section of said one coil line turn facing an inner diameter side of said sheath body, and an upper short side of said trapezoidal cross section of said other coil line turn facing an outer diameter side of said sheath body;
one oblique side of said trapezoidal cross section of said one coil line turn and one oblique side of said trapezoidal cross section of said other coil line turn being in contact with each other to form a mutual neighborhood side; and
oblique sides of neighboring trapezoidal cross sections being relatively slidable each other at said mutual neighborhood side in a radial direction of said sheath body when said sheath body is bent.

3. In a medical treatment equipment which includes a treatment portion provided at a distal end portion, an operational portion provided at a proximal portion, an elongate core provided to connect said treatment portion to said operational portion, and an elongated flexible sheath body, to which said elongate core is inserted;
said medical treatment equipment comprising:
said sheath body having a deformed tight coil structure in which a plurality of wire elements formed triangular in cross section and a plurality of wire elements formed trapezoidal in cross section are helically wound with one coil line turn alternately arranged in neighborhood of other coil line turn;
an upper short side of said trapezoidal cross section of said one coil line turn facing an inner diameter side of said sheath body, and an apex of said triangular cross section of said other coil line turn facing an outer diameter side of said sheath body;
one oblique side of said trapezoidal cross section of said one coil line turn and one side of said triangular cross section of said other coil line turn being in contact with each other to form a mutual neighborhood side; and
said one oblique side of said trapezoidal cross section and said one side of said triangular cross section being relatively slidable each other at said mutual neighborhood side in a radial direction of the said sheath body when said sheath body is bent.

4. The medical treatment equipment according to claim 1 or 2, wherein cross sectional shapes of said plurality of wire elements are analogous each other.

5. The medical treatment equipment according to claim 1 or 2, wherein cross sectional shapes of said plurality of wire elements are identical each other.

6. The medical treatment equipment according to any one of claims 1-3, wherein cross sectional shapes of said plurality of wire elements have equilateral oblique sides, and angles of said equilateral oblique sides are 45 degrees.

7. The medical treatment equipment according to any one of claims 1-3, wherein said treatment portion is a biopsy cup which is operated by a push-pull manipulation through said operational portion for collecting cellular tissues.

8. The medical treatment equipment according to any one of claims 1-3, wherein said treatment portion is a looped wire which is tightened to be diametrically reduced by pulling said elongate core through said operational portion for tying up cellular tissues, so as to form an endoscopic treatment structure.

9. The medical treatment equipment according to any one of claims 1-3, wherein said treatment portion is a clip retainer which accommodates a clip within said sheath body and retains said clip in cellular tissues by pulling said elongate core through said operational portion, so as to form a multi-functional surgical treatment structure.

10. The medical treatment equipment according to any one of claims 1-3, wherein said treatment portion is an endoscope-angle shifting portion which is manipulatively altered by adjusting a tension of said elongate core through a rotational movement of said operational portion so as to form an endoscopic structure.

11. The medical treatment equipment according to any one of claims 1-3, wherein a sensor is built in said sheath body to form a sensor-attached guide wire structure.

12. The medical treatment equipment according to any one of claims 1-3, wherein said sheath body has a dual region structure which consists of a distal end side region having said deformed tight coil structure and a proximal end side region having a wire-stranded hollow coil structure connected in series to a proximal side of said distal end side region.

13. The medical treatment equipment according to any one of claims 1-3, wherein said sheath body has a triple region structure which consists of a distal end side region having said deformed tight coil structure, an intermediary region having a wire-stranded hollow structure connected in series to a proximal side of said distal end side region, and a proximal end side region having a tubular structure connected in series to a proximal side of said intermediary region.
